(19) United States
(12) Patent Application Publication
(10) Pub. No.: US 2009/0107495 A1
(43) Pub. Date: Apr. 30, 2009

(54) DEVICE FOR INHALATION OF MEDICINE

(75) Inventors: Henmei Ni, Ibaraki (JP); Yoshiyuki Uchida, Ibaraki (JP); Wataru Kamimura, Ibaraki (JP); Junzo Tanaka, Ibaraki (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: NATIONAL INSTITUTE FOR MATERIALS SCIENCE, Tsukuba-shi, Ibaraki (JP)

(21) Appl. No.: 11/996,431

(22) PCT Filed: Jul. 21, 2006
(86) PCT No.: PCT/JP2006/314504

§ 371 Date: Sep. 23, 2008

(30) Foreign Application Priority Data
Jul. 21, 2005 (JP) 2005-211809

(51) Int. Cl. A61K 9/51; A61M 16/06; B29C 47/04

(52) U.S. Cl. 128/203.15; 424/489; 264/465

(57) ABSTRACT

An object of the present invention is to provide an inhalation device by which nano-sized particles of a medicine are able to be stably present without aggregation even in air or the like where no dispersing solvent is present. A device for inhalation of medicine according to the present invention for solving such a problem is that where medicine particles in a nano- to micron-size are adhered to a matrix comprising a nano fiber to such an extent that the particles are detached by inspiration permeating through the matrix.

### TECHNICAL FIELD

The present invention relates to an inhalation device for medicine particles in nano- to micron-sizes.

### BACKGROUND ART

It has been well known that inhalation preparations of medicines have been already used in the treatment of respiratory diseases such as bronchial asthma. Particle size of the medicine in an inhalation preparation which has been actually used in the market is in several microns, and it is designed in such a manner that the medicine is mostly sedimented within an area from bronchus to bronchiole. In recent years, development of inhalation preparations of insulin for the treatment of diabetes mellitus has been also carried out. Size of the insulin particles in the insulin inhalation preparation is also in several microns.

Particle distribution of a medicine in the lung is able to be changed by adjusting its particle size. Micron-sized particles sediment in the lung and the airway as mentioned above, while nano-sized particles by-pass said area and are able to be directly dissolved in the artery. Accordingly, an inhalation preparation of nano-sized particles of a medicine is expected to effectively act on various kinds of systemic diseases in a small dose, and that is a desirable means in medical economy as well.

However, although nano-sized particles of a medicine is able to be stably present in a dispersing solvent, there is a characteristic that they become a big cluster causing aggregation in air, etc. where no dispersing solvent is present whereby characteristic of each nano-sized particle disappears. Accordingly, unless any method for solving such a problem will be found, it will be difficult to put a preparation by which inhalation of nano-sized particles of a medicine is possible to practical use.

### DISCLOSURE OF THE INVENTION

### Problems that the Invention is to Solve

Under such circumstances, an object of the present invention is to provide an inhalation device by which nano-sized particles of a medicine are able to be stably present without aggregation even in air or the like where no dispersing solvent is present.

### Means for Solving the Problems

In view of the above, the present inventors have carried out intensive studies repeatedly and, as a result, they have found that, when a solution containing a medicine is spray-dried onto the surface of a matrix comprising a nano fiber, medicine particles in a nano- to micron-size are adhered on the surface of the nano fiber without aggregation of the particles in such a strength that the particles are detached by the pressure upon inspiration which permeates through the matrix.

A device for inhalation of medicine according to the present invention which has been achieved on the basis of the above finding is that, as mentioned in claim 1, medicine particles in a nano- to micron-size are adhered to a matrix comprising a nano fiber to such an extent that the particles are detached by inspiration permeating through the matrix.

The device for inhalation of medicine mentioned in claim 2 is that, in the device for inhalation of medicine mentioned in claim 1, the nano fiber comprises a macromolecular material.

The device for inhalation of medicine mentioned in claim 3 is that, in the device for inhalation of medicine mentioned in claim 2, the macromolecular material is polyvinylpyrrolidone or polyvinylalcohol.

The device for inhalation of medicine mentioned in claim 4 is that, in the device for inhalation of medicine mentioned in any of claims 1 to 3, the matrix comprising a nano fiber is formed on the surface of a fiber carrier.

The device for inhalation of medicine mentioned in claim 5 is that, in the device for inhalation of medicine mentioned in claim 4, the fiber carrier is an organic fiber carrier.

The device for inhalation of medicine mentioned in claim 6 is that, in the device for inhalation of medicine mentioned in claim 5, the organic fiber carrier is a cotton mat.

A method for the manufacture of the device for inhalation of medicine according to the present invention is that, as mentioned in claim 7, a solution containing a medicine is spray-dried onto a matrix comprising a nano fiber whereby medicine particles in a nano- to micron-size are adhered on the surface of the nano fiber to such an extent that the particles are detached by inspiration permeating through the matrix.

The method for the manufacture mentioned in claim 8 is that, in the method for the manufacture mentioned in claim 7, an electrospinning by using a solution of a macromolecule as a starting material is carried out to form a matrix comprising a nano fiber.

The method for the manufacture mentioned in claim 9 is that, in the method for the manufacture mentioned in claim 8, the matrix comprising a nano fiber is formed on the surface of a fiber carrier.

The method for the manufacture mentioned in claim 10 is that, in the method for the manufacture mentioned in claim 9, the fiber carrier is placed on an electroconductive metal foil and an electrospinning is carried out by applying voltage between a nozzle and the electroconductive metal foil to form a matrix comprising a nano fiber which comprises a macromolecular material on the surface of the fiber carrier.

A mask for inhalation of medicine according to the present invention is that, as mentioned in claim 11, a device for inhalation of medicine that medicine particles in a nano- to micron-size are adhered to a matrix comprising a nano fiber and formed on the surface of a fiber carrier to such an extent that the particles are detached by inspiration permeating through the matrix is made into a shape of a mask.

### ADVANTAGES OF THE INVENTION

In accordance with the present invention, it is now possible to provide an inhalation device by which nano-sized particles of a medicine are able to be stably present without aggregation even in air or the like where no dispersing solvent is present whereby practical implementation of a pharmaceutical preparation by which inhalation of nano-sized particles of a medicine is possible is now made easy.

### BRIEF DESCRIPTION OF THE DRAWINGS

FIG. 1 is a picture, under a scanning electron microscope, of a cotton mat where a matrix comprising a nano fiber of PVP in which creatine particles are adhered on the surface is formed on the surface in Examples.

FIG. 2 is a picture, under a scanning electron microscope, after the creatine particles are detached by air stream in Examples.

### BEST MODE FOR CARRYING OUT THE INVENTION

A device for inhalation of medicine according to the present invention is that medicine particles in a nano- to micron-size are adhered to a matrix comprising a nano fiber to such an extent that the particles are detached by inspiration permeating through the matrix (velocity is 1 to 25 L/minute for example). An example of the matrix comprising a nano fiber is a matrix which is formed by a macromolecular material. As to an appropriate macromolecular material, although there may be listed polyvinylpyrrolidone and polyvinylalcohol which are water soluble and do not afford bad influence even when inhaled into human body with medicine, the material may be a polyamino acid such as gelatin or may be a polysaccharide such as cellulose as well. As to the macromolecular material, a single material may be used or plural types thereof may be mixed and used. A matrix comprising a nano fiber which comprises a macromolecular material may, for example, be formed by subjecting a solution of a macromolecule as a starting material to an electrospinning which has been known per se.

As to an appropriate method for adhesion of medicine particles in a nano- to micron-size on the surface of the nano fiber to such an extent that the particles are detached by inspiration permeating through a matrix, there may be listed a method where a solution containing a medicine is spray-dried by a known method per se onto a matrix comprising a nano fiber.

With regard to a matrix comprising a nano fiber where medicine particles in a nano- to micron-size are adhered on the surface in such a manner that the particles are detached by inspiration permeating therethrough, the matrix per se may be made into various forms (such as nonwoven fabric, woven fabric, sheet or mat) and shapes and is used as a device for inhalation of medicine. However, if it is formed on the surface of a fiber carrier, a mask for inhalation of medicine where the medicine particles in a nano- to micron-size adhered on the surface of the nano fiber are detached by inspiration and inhaled into human body is able to be easily manufactured when the matrix formed on the surface of the fiber carrier is made into a shape of a mask. The fiber carrier may comprise either an organic fiber or an inorganic fiber. Examples of the organic fiber are a natural fiber such as cotton, silk and linen and a synthetic fiber such as nylon, polyethylene terephthalate, polypropylene, polyethylene and polystyrene. Examples of the inorganic fiber are ceramic fiber, glass fiber and fiber made of metal such as iron and aluminum. The fiber carrier may be a composite fiber comprising any combination of an organic fiber and an inorganic fiber or may be a network structure. As to the fiber carrier, an organic fiber carrier or, particularly, cotton mat may be advantageously adopted when safety to human body, easiness in processing, cost, etc. are taken into consideration. Formation of the matrix comprising the nano fiber which comprises a macromolecular material on the surface of the fiber carrier may be efficiently carried out by placing the fiber carrier on an electroconductive metal foil (such as aluminum foil) and by conducting an electrospinning by applying voltage between a nozzle and the electroconductive metal foil.

### EXAMPLES

The present invention will now be illustrated in detail by way of the following Examples, although the present invention is never interpreted in a limited manner by the following descriptions.

### Example 1

A commercially available electrospinning apparatus (HSP-30K-2 manufactured by Nippon Stabilizer Industry Co., Ltd.) was used, voltage (direct current of 15 kV) was applied between (the distance being 20 cm) a nozzle and an aluminum foil (manufactured by Mitsubishi Aluminum Company, Ltd.) on which a cotton mat (manufactured by Asahi Kasei Corporation) is placed attached to a collector of the apparatus and a 10 wt % ethanolic solution of polyvinylpyrrolidone (PVP) was sprayed at ambient temperature from the nozzle onto the cotton mat to form a matrix comprising a nano fiber of PVP on the surface of the cotton mat. A fiber diameter of the nano fiber of PVP constituting the matrix was measured by using a scanning electron microscope (SEM JSM-5400 manufactured by JOEL Ltd.) and found to be from 300 to 700 nm.

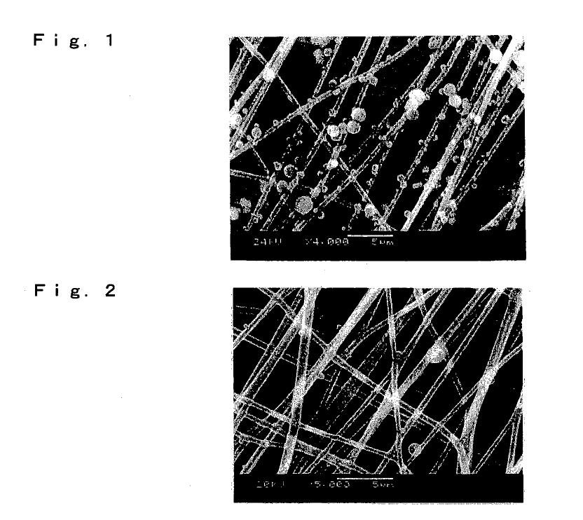

The cotton mat where a matrix comprising a nano fiber of PVP was formed on the surface was attached onto a filter of a commercially available spray-drying apparatus (Buchi Mini Spray Drier B-290 manufactured by Nihon BUCHI K.K.) and a 1 wt % aqueous solution of creatine was spray-dried onto the matrix (outlet temperature of the nozzle: 180° C.; aspirator speed: 35%; pump speed: 5%) to give a cotton mat where a matrix comprising a nano fiber of PVP in which creatine particles were adhered on the surface was formed on the surface. A picture of the cotton mat taken under a scanning electron microscope (the same one as mentioned already) is shown in FIG. 1. As will be apparent from FIG. 1, the particle size of creatine adhered on the surface of the nano fiber of PVP was about 2 μm at the largest and many of the particles were in a size of from 100 nm to smaller than 1 μm.

The cotton mat where the matrix comprising the nano fiber of PVP in which creatine particles were adhered on the surface was formed on the surface was exposed for 1 minute in air stream having the similar degree as human inspiration (velocity is 5 L/minute). A picture of the cotton mat after exposure taken under a scanning electron microscope (the same one as mentioned already) is shown in FIG. 2. As will be apparent from FIG. 2, the creatine particles adhered on the surface of the nano fiber of PVP were detached by air stream. Therefore, it was noted that, according to this method, creatine particles in a nano- to micron-size were stably retained even in air and that they were able to be inhaled into human body without losing the characteristics thereof by inspiration.

### Example 2

A cotton mat where a matrix comprising a nano fiber of PVP in which creatine particles in a nano- to micron-size were adhered on the surface to such an extent that the particles were detached by inspiration permeating therethrough was formed on the surface prepared in Example 1 was made into a shape of a mask to prepare a mask for inhalation of creatine.

### Example 3

A cotton mat where a matrix comprising a nano fiber of PVA in which creatine particles in a nano- to micron-size were adhered on the surface to such an extent that the particles were detached by inspiration permeating therethrough was formed on the surface was prepared by the same manner as in Example 1 except that a 10 wt % ethanolic solution of polyvinylalcohol (PVA) was used in place of a 10 wt % ethanolic solution of polyvinylpyrrolidone (PVP).

### Example 4

A cotton mat where a matrix comprising a nano fiber of PVP in which particles of a nano- to micron-sized composite of Ebselen (2-phenyl-1,2-benzoisoselenazol-3-(2H)-one), which is an antioxidant substance, with N-acetylcysteine were adhered on the surface to such an extent that the particles were detached by inspiration permeating therethrough was formed on the surface was prepared by the same manner as in Example 1 except that, in place of a 1 wt % aqueous solution of creatine, a 10 wt % ethanolic solution of a composite of Ebselen with N-acetylcysteine (although Ebselen was hardly soluble in ethanol, its solubility is able to be made to an extent of about 100-fold by making it into a composite with N-acetylcysteine) was used.

### Example 5

A cotton mat where a matrix comprising a nano fiber of PVP in which Ebselen particles in a nano- to micron-size were adhered on the surface to such an extent that the particles were detached by inspiration permeating therethrough was formed on the surface was prepared by the same manner as in Example 1 except that a 10 wt % DMSO solution of Ebselen was used in place of a 1 wt % aqueous solution of creatine.

### INDUSTRIAL APPLICABILITY

The present invention has an industrial applicability since it is able to provide an inhalation device by which nano-sized particles of a medicine are able to be stably present without aggregation even in air or the like where no dispersing solvent is present.

1. A device for inhalation of medicine where medicine particles in a nano- to micron-size are adhered to a matrix comprising a nano fiber to such an extent that the particles are detached by inspiration permeating through the matrix.

2. The device for inhalation of medicine according to claim 1, wherein said nano fiber comprises a macromolecular material.

3. The device for inhalation of medicine according to claim 2, wherein said macromolecular material is polyvinylpyrrolidone or polyvinylalcohol.

4. The device for inhalation of medicine according to any one of claims 1 to 3, wherein said matrix comprising a nano fiber is formed on the surface of a fiber carrier.

5. The device for inhalation of medicine according to claim 4, wherein said fiber carrier is an organic fiber carrier.

6. The device for inhalation of medicine according to claim 5, wherein said organic fiber carrier is a cotton mat.

7. A method for the manufacture of the device for inhalation of medicine where a solution containing a medicine is spray-dried onto a matrix comprising a nano fiber whereby medicine particles in a nano- to micron-size are adhered on the surface of the nano fiber to such an extent that the particles are detached by inspiration permeating through the matrix.

8. The method for the manufacture according to claim 7, wherein an electrospinning by using a solution of macromolecule as a starting material is carried out to form a matrix comprising a nano fiber.

9. The method for the manufacture according to claim 8, wherein, said matrix comprising a nano fiber is formed on the surface of a fiber carrier.

10. The method for the manufacture according to claim 9, wherein said fiber carrier is placed on an electroconductive metal foil and an electrospinning is carried out by applying voltage between a nozzle and the electroconductive metal foil to form a matrix comprising a nano fiber which comprises a macromolecular material on the surface of said fiber carrier.

11. A mask for inhalation of medicine where a device for inhalation of medicine that medicine particles in a nano- to micron-size are adhered to a matrix comprising a nano fiber and formed on the surface of a fiber carrier to such an extent that the particles are detached by inspiration permeating through the matrix is made into a shape of a mask.
